(19) United States
(12) Patent Application Publication
(10) Pub. No.: US 2005/0014690 A1
(43) Pub. Date: Jan. 20, 2005

(54) Styryl acrylonitrile compounds and their use to promote myelopoiesis

(76) Inventors: Chaim Roifman, Ontario (CA); Thomas Grunberger, Ontario (CA); Nigel Sharfe, Toronto (CA); Olga Rounova, Toronto (CA); Demin Peter, Toronto (CA); Andrew Freywald, Thornhill (CA)

Correspondence Address: ROPES & GRAY LLP, ONE INTERNATIONAL PLACE, BOSTON, MA, 02110-2624, US

(21) Appl. No.: 10/492,251

(22) PCT Filed: Oct. 11, 2002
(86) PCT No.: PCT/CA02/01548

(30) Foreign Application Priority Data
Oct. 11, 2001 (US) 60329168

(51) Int. Cl. A61K 38/17

(52) U.S. Cl. 514/12

(57) ABSTRACT

The invention relates to the use of organic compounds, styryl acrylonitrile compounds to promote myelopoiesis. These compounds thefore may be used to treat a subject suffering from neutropenia and other conditions which would benefit from increased myelopoieses. Moreover, the compounds may be used to treat hematopoietic cells ex-vivo to promote myelopoiesis and therefore may be used advantageously in bone marrow or peripheral blood stem cell transplant.

### RELATED APPLICATION

This application claims the benefit of U.S. application Ser. No. 60/329,168 filed Oct. 11, 2001.

### FIELD OF THE INVENTION

This invention relates to the use of therapeutic organic compounds to promote the proliferation and/or differentiation of hematopoietic cells, in particular cells of the myeloid lineage and those giving rise to the myeloid lineage.

### BACKGROUND OF THE INVENTION

Granulocytes or polymorphonuclear leukocytes (PMNL) (neutrophils, basophils, and eosinophils), and macrophages of the innate immune system provide an important defense against infections. These cells, like all other cellular elements of blood arise from hematopoietic stem cells in the bone marrow. More particularly, they are leukocytes derived from the myeloid stem (also termed myeloid progenitor) cell resulting from initial differentiation of hematopoietic stem cells.

Neutropenia and its Current Treatment

Neutrophils are the most common leukocyte in the peripheral blood of healthy persons, and circulate from bone marrow to peripheral blood and into tissues. These circulating and tissue pools are in a dynamic equilibrium, with neutrophils oscillating between them. Neutrophils play an integral role in the host defense against potential bacterial and opportunistic fungal pathogens. During infection, neutrophils in the blood are attracted to sites of infection by chemotactic factors generated by the interaction of host cells with pathogens. Neutrophil disorders are a relatively uncommon and yet an important cause of morbidity and mortality in infants and children (1, 2).

There are many examples of clinical conditions characterized by neutropenia, or a lack of neutrophils, both primary and secondary (3-5). These include, but are not restricted to deficiencies created by anti-cancer treatments, by hematological malignancies, by drugs, autoimmune neutropenia and mutations in hematopoietic growth factor receptors. In particular, the expanding use of dose-intensive cancer treatment strategies, such as high-dose chemotherapy and bone marrow and hematopoietic stem cell transplantation, has increased the frequency of prolonged neutropenia and, consequently, the risk of severe infections in affected patients. Infection is one of the most serious complications of cancer therapy. In general, opportunistic fungal infections and antibiotic-refractory bacterial infections remain important causes of morbidity and mortality in neutropenic individuals (6). Although gram negative bacteria predominate in the early infections observed in neutropenic patients, fungal infections (Aspergillus, Zygomycetes, Fausarium species) become common with persistence of the neutropenic condition. While “opportunistic” fungi such as Candida and Aspergillus species, may only induce occasional allergic phenomena in a healthy individual, they produce far more invasive diseases in immuno-suppressed patients.

Distinct from the induced secondary neutropenias, congenital neutropenia is a group of hematopoietic disorders characterized by a profound, absolute neutropenia due to a maturational arrest of myeloid progenitor cells. About 10% of patients undergo malignant progression associated with acquired nonsense mutations in the Granulocyte-Colony Stimulating Factor (G-CSF) receptor. Mutations in the G-CSF receptor in congenital neutropenia are most probably connected with the progression of the neutropenia to myelodysplastic syndrome (MDS)/leukemia as the result of a loss of differentiation signaling (7, 8).

Recombinant hematopoietic growth factors have come to enjoy widespread use in both pediatric and adult oncology, to reduce morbidity from chemotherapy regimes (9-11). In the standard treatment of patients with hematological malignancy, immunosuppressive therapy produces long periods of neutropenia, with increased risk of fungal infection. The neutropenia caused by many anti-cancer drugs is commonly a limiting factor in dose escalation. Currently, recombinant G-CSF is widely used therapeutically for the treatment of neutropenia, including neutropenia secondary to chemotherapy, radiotherapy, or myelosuppressive drugs, as well as leukemia, idiopathic neutropenia, and aplastic anemia. The number and function of PMNL are regulated by cytokines, especially G-CSF. In the laboratory G-CSF deficient mice develop chronic neutropenia associated with a 50% reduction of PMNL precursor cells in the bone marrow (12, 13). These mice exhibit a markedly impaired ability to control infection by Listeria monocytogenes and do not develop sepsis-related neutrophilia. On the basis of trials conducted in patients with chemotherapy-induced neutropenia, G-CSF (and GM-CSF (Granulocyte-macrophage-CSF)) has been widely used for the acceleration of marrow recovery after standard-dose chemotherapy. Both G-CSF and GM-CSF have been shown in numerous trials to shorten the period of chemotherapy induced neutropenia, with a reduction in the attendant morbidity and to mobilize peripheral blood stem cells. They can significantly reduce the requirement for antibiotics and the duration of hospitalization. However, both demonstrate drawbacks. GM-CSF displays an extremely narrow therapeutic window and G-CSF is less effective in patients with severe neutropenia resulting from dose-intensive chemotherapy due to a lack of G-CSF responsive hematopoietic progenitors. Several weeks of severe neutropenia is not unusual in these patients, and life-threatening bacterial or fungal infections remain a substantial problem. Recombinant growth factors are also difficult to make and expensive as a result.

Transfusion of large numbers of neutrophils has also been examined as a clinical solution to neutropenia (14, 15). However, its usefulness for the treatment or prevention of fungal and bacterial infections remains controversial. Insufficient donor stimulation regimens and suboptimal leukapheresis techniques have long limited the clinical benefits of the procedure. However, the ability to mobilize neutrophils in healthy donors by administration of G-CSF has significantly enhanced leukapheresis yields and several studies suggest that it may be possible to store functional granulocyte concentrates for 24 to 48 hours. However, the necessity for donor mobilization and concerns over blood product donation suggests that this will not ultimately provide a satisfactory solution.

### SUMMARY OF THE INVENTION

The present invention is based on the unexpected discovery that the acrylonitrile compounds (EE)-2-(benzylaminocarbonyl)-3-(3,4-dihydroxystyryl)acrylonitrile(CR4), (E,E)-2-cyano-3-(3,5-dimethoxy-4-hydroxystyryl)acrylonitrile (CR7), (E,E)-2-(phenylethylamido)-3-(3,5-dimethoxy-4-hydroxystyryl)acrylonitrile (CR8), (E,E)-2-(3,4-dihydroxybenzylaminocarbonyl)-3-(3,5-dimethoxy-4-hydroxystyryl)acrylonitrile (CR11), E,E)-2-(3,4 dihydroxybenzylaminocarbonyl)-3-styrylacrylonitrile (CR19) and (E,E)-2-(benzylaminocarbonyl)-3-(3-methoxy-4-hydroxystyryl)acrylonitrile (CR56) are capable of promoting myelopoiesis (defined herein has proliferation and/or differentiation of cells of the myeloid lineage including granulocytes, monocytes and its differentiated form, macrophages), in vitro, ex vivo and in vivo. The ability of these compounds to promote myelopoiesis was unexpected. These compounds are referred to in this application as CR compounds and by reference to a CR compound is meant a compound selected from this group. CR4, CR8, CR11 and CR19 are described in copending U.S. application Ser. No. 09/834,728, the contents of which are hereby incorporated by reference.

In its broadest aspect, the invention relates to a method of promoting myelopoeisis in vivo, ex vivo and in vitro.

In one aspect, the invention relates to a method of promoting myelopoiesis comprising administering an effective amount of a CR compound to hematopoietic cell or an animal in need thereof. The term “cell” includes a plurality of cells. Administration to a cell includes in vivo, ex vivo and in vitro treatment. In specific embodiments, the CR compound is CR4, CR11 or CR19.

In some embodiments, the hematopoietic cell is hematopoeitic stem cell and the animal is a human patient. In specific embodiments, the compounds are administered to a human patient suffering from, or at risk of primary or secondary neutropenia, including chemotherapy or drug induced neutropenia, neutropenia secondary to malignancy, including G-CSF responsive malignancies. In other embodiments, the compounds are administered to a human patient at risk of, or suffering from aplastic anemia or aplasia. In other embodiments, the animal is a human donor of bone marrow cells or peripheral blood stem cells. In other embodiments, CR4, CR11, or CR19 is administered to a human patient in need of bone marrow cell or peripheral blood stem cell transplant before or after the transplant.

In another aspect, the invention provides a method of promoting myelopoiesis ex vivo comprising administering an effective amount of a CR compound to hematopoietic cell. In specific embodiments, the CR compound is CR4, CR11 or CR19. In one embodiment, the cell is hematopoietic stem cell. In specific embodiments, the hematopoietic cell is from the bone marrow or peripheral blood stem cells of a donor, or the bone marrow or peripheral blood stem cells of a patient in need of autologous bone marrow or peripheral blood stem cell transplant.

In another aspect, the invention provides a method of treating a patient suffering from or at risk of neutropenia, aplastic anemia or aplasia comprising administering an effective amount of a CR compound to said patient. In specific embodiments, the CR compound is CR4, CR11 or CR19. In another aspect, the invention provides a method of treating a patient suffering from or at risk of neutropenia, aplastic anemia or aplasia comprising introducing hematopoietic cells to the patient wherein a CR4 compound has been administered to the cells ex vivo in an amount effective to promote myelopoiesis. In specific embodiments, the CR compound is CR4, CR11 or CR19. The hematopoietic cells may be from the bone marrow or peripheral blood stem cells of a donor or of the patient.

In another aspect, the invention relates to use of a CR compound to promote myelopoiesis. The invention also relates to use of a CR compound for preparing a medicament to promote myelopoiesis. Yet in another aspect, the invention relates to use of a CR compound to treat neutropenia, aplastic anemia or aplasia, and the use of a CR compound to prepare a medicament to treat neutropenia aplastic anemia or aplasia. In specific embodiments, the CR compound is CR4, CR11 or CR19.

In another aspect, the invention provides a kit comprising a CR compound and instructions for use, including to promote myelopoiesis and to treat neutropenia, aplastic anemia and aplasia. In specific embodiments, the CR compound is CR4, CR11 or CR19.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 is a graph demonstrating the promotion of normal bone marrow myelopoiesis by CR4 on long term exposure.

FIG. 2 is a graph demonstrating the promotion of myelopoiesis of CD34+ multipotent hematopoietic stem cell by CR4 on long term exposure.

FIG. 3 is a graph demonstrating the dose response of CR4 promotion of normal bone marrow myelopoiesis.

FIG. 4 is a graph demonstrating the promotion of normal bone marrow myelopoiesis by CR11.

FIG. 5 is a graph demonstrating the promotion of normal bone marrow myelopoiesis by CR4 after 2.5 hours exposure.

FIG. 6 is a graph demonstrating the promotion of normal bone marrow myelopoiesis by CR4 after different exposure times.

FIG. 7 is a graph demonstrating the dose response of CR4 promotion of normal bone marrow myelopoiesis after five hours exposure.

FIG. 8, is a graph demonstrating the promotion of normal bone marrow myelopoiesis by CR19.

FIG. 9, is a graph demonstrating an increase in peripheral blood white blood cell counts with administration of G-CSF and CR11 to mice.

FIG. 10 is a photograph demonstrating an increase in granulocytes in the spleen of CR4 treated mice compared to a normal spleen.

FIGS. 11 and 12 demonstrate effects of CR4 on normal bone marrow myelopoiesis in the long-term culture (FIG. 11) and in the short-term CFU-GEMM assay (FIG. 12).

### DETAILED DESCRIPTION OF THE INVENTION

CR compounds can promote the proliferation and or differentiation of hematopoietic cells of the myeloid lineage, in vitro, ex vivo, and in vivo.

By way of specific examples, CR4, CR11 and CR19 are effective in increasing the number of CFU-GM (colony forming unit-granulocyte and macrophage) or CFU-C (granulocyte and monocyte and macrophage) colonies in in vitro assays of human bone marrow cell growth. This promotion of myelopoiesis may be obtained under conditions of continual exposure for 14 days to low levels of the compounds (about 1-20 μM), with both whole normal bone marrow cells and purified CD34⁺ hamatopoietic stem cells. When normal bone marrow cells were incubated in the presence of CR4 or CR11 for a period of 14 days minimal toxicity was observed until concentrations of 20 μM or greater.

The effect may also be obtained by short periods of exposure of hematopoietic cells to higher concentrations of the CR compounds. Higher doses of CR4 (25-50 μM) administered to bone marrow cells for 2-5 hours results in increased myelopoiesis when the cells are cultured. Other normal bone marrow cells exposed to the same doses over the same period of time remain relatively unaffected. Therefore, the CR compounds may be administered to hematopoietic cells to promote myelopoiesis ex vivo. For example, CR4, CR11 and CR19 may be administered to promote myelopoiesis ex vivo by administering CR4, CR11 or CR19 to bone marrow cells or peripheral blood stem cells removed from a patient suffering from neutropenia, and the cells reintroduced into the patient to promote the recovery of immune function. The bone marrow cells or peripheral blood stem cells may also be obtained from a donor, treated with CR4, CR11 or CR19 and then introduced to a patient suffering from neutropenia. Ex vivo treatment is advantageous in a number of respects. The period of cell treatment required to achieve a significant increase in myelopoiesis is short. Minimal manipulation of the patient bone marrow is required. Moreover, ex vivo treatment of the cells reduces the risk of any adverse side effects.

The CR compounds, including CR4, CR11 or CR19 may also be administered in vivo to promote myelopoiesis. In an in vivo murine model, daily injection of CR4 or CR11 into a SCI mouse model revealed a significant increase in myelopoiesis, as evidenced by a tremendous increase in normal splenic granulocytes. Increased numbers of white blood cells were also observed in the peripheral blood, appearing with similar kinetics to increases in G-CSF treated mice. The doses required to promote myelopoiesis did not result in detectable non-specific damage to the animal over a period of one month of continual administration.

Furthermore, long-term myelopoiesis may be achieved by administering a CR compound, including CR4, CR11 or CR19. Normal bone marrow cells when treated with 50 μM of CR4 for 5 hours demonstrated a significant increase in the number of CFU-GM in long-term cultures.

Given this combination of highly specific, long-term effect upon myelopoiesis and minimal non-specific cytotoxic damage, a CR compound may be advantageously used ex vivo or in vivo to promote proliferation and stimulation of hematopoietic cells of myeloid lineage. In one embodiment, the CR compound is CR4, CR11 or CR19.

As such, a CR compound may be used to treat all conditions which would benefit from increased myelopoiesis, including neutropenia, aplastic anemia and aplasia and other conditions currently treated with G-CSF. It is well accepted that small synthetic molecules offer advantages related to relative ease of synthesis, cost, and stability. Additionally, relative to biological agents such as recombinant G-CSF, these molecules may produce fewer side effects due to more limited scope of action and at the same time, have therapeutic effect on cells that may not express receptors for the biological agents.

In specific embodiments, CR4, CR11 and CR19 may be administered to promote myelopoiesis in human patients suffering from or at risk of primary or secondary neutropenia.

The CR compounds may be particularly effective in restoring immune function in patients suffering from chemotherapy or drug induced neutropenia. Accordingly, in one embodiment, CR4, CR11, and CR19 are administered to a patient suffering from or at risk of chemotherapy or drug induced neutropenia. These patients often require bone marrow cell or peripheral blood stem cell transplant (autologous or non-autologous) and in one embodiment, the compounds may be administered to a patient before, or after bone marrow cells or peripheral blood stem cells are introduced into the patient, to promote myelopoiesis of the transplanted cells and thereby promote the restoration of normal immune function in the patient.

A CR compound may also be administered to a patient suffering from, or at risk of neutropenia secondary to malignancy. CR4, CR11 and CR19 may be of particular utility in cases of neutropenia secondary to G-CSF responsive malignancies, where treatment with G-CSF is clearly undesirable due to the potential promotion of residual tumor cell growth. CR4, CR11 and CR19 do not promote transformed cell growth.

Currently, G-CSF is administered to donors of bone marrow cells to increase the number of activated hematopoietic cells. A CR compound, preferably CR4, CR11 or CR19, in one embodiment may also be administered to donors of hematopoietic cells including, bone marrow cells prior to harvesting to increase the number of hematopoietic cells of the myeloid lineage. In other embodiments, the compounds may be administered ex vivo to bone marrow cells of a donor prior to their transfer to a patient in need thereof. Autologous bone marrow transplant samples could be similarly treated such that in one embodiment, the compounds are administered ex vivo to bone marrow cells of a patient in need of autologous bone marrow cell transplant.

G-CSF-mobilized peripheral blood stem cells are now widely used instead of bone marrow cells for transplantation in patients with advanced hematologic malignancies, due to earlier hematopoietic recovery after transplant, with lowered transplant-related mortality and fewer relapses as a result of improved immune reconstitution and a graft-versus-leukemia effect. A CR compound such as CR4, CR11, and CR19 therefore may also be administered to a donor of peripheral blood stem cells prior to harvesting or administered ex-vivo to peripheral blood stem cells of a donor. The compounds may also be administered ex-vivo to peripheral blood cells of a patient in need of autologous peripheral blood stem cell transplantation.

Aplastic anemia and inherited or acquired aplasias are treated with G-CSF to restore immunological function by increasing differentiation of cells of hematopoeitic lineages. In one embodiment, CR4, CR11 or CR19 are administered to patient at risk of or suffering from aplastic anemia or aplasia.

The compounds may be used in the form of the free base, or in other forms such as salts, prodrugs, solvates, and hydrates, and reference to a CR compound is intended to encompass all such forms of the compound. The acids which can be used to prepare acid addition salts are those which produce, when combined with the compound, pharmaceutically acceptable salts, that is, salts whose anions are non-toxic to the animal in pharmaceutical doses of the salts, so that the beneficial properties inherent in the free base are not vitiated by side effects ascribable to the anions. Pharmaceutically acceptable salts include those derived from the following acids; mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexysulfamic acid, quinic acid, and the like.

Similarly, basic addition salt may be prepared using an inorganic base such as lithium, sodium, potassium, calcium, magnesium or barium hydroxide. Illustrative organic bases which form suitable salts include aliphatic, alicyclic or aromatic organic amines such as methylamine, trimethylamine and picoline or ammonia.

The selection of other pharmaceutically acceptable salts will be known to a person skilled in the art and a desired salt may be prepared using standard techniques.

Prodrugs of the compounds may be conventional esters formed with available hydroxy, amino or carboxyl group on the compound. For example, an OH group may be acylated using an activated acid in the presence of a base, and optionally, in inert solvent (e.g. and acid chloride in pyridine). Some common esters which have been utilized as prodrugs are phenyl esters, aliphatic (C₈-C₂₄) esters, acyloxymethyl esters, carbamates and amino acid esters. Conventional procedures for the selection and preparation of suitable prodrugs are described, for example, in “Design of Prodrugs” ed. H. Bundagaard, Elsevier, 1985.

A “solvate” is formed when a suitable solvent are incorporated in the crystal lattice of the compound or salt thereof. A suitable solvent is physiologically tolerable at the dosage administered. Examples of suitable solvents are ethanol, water and the like. When water is the solvent, the molecule is referred to as a “hydrate”. Methods to prepare a solvate are known in the art. In general, solvates are prepared by dissolving the compound in the appropriate solvent and isolating the solvate by cooling or using an antisolvent. The solvent is typically dried or azeotroped under ambient conditions.

For administration in vivo, the compounds are preferably formulated into pharmaceutical compositions in a biologically compatible form suitable. Accordingly, in one embodiment, a CR compound is administered to a human patient in combination with a pharmaceutically acceptable carrier.

The compositions containing a CR compound can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffer solutions with a suitable pH and iso-osmotic with the physiological fluids.

The compounds may be administered to a patient in a variety of forms depending on the selected route of administration, as will be understood by those skilled in the art. The compositions of the invention may be administered orally or parenterally. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

The compounds may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound of the invention may be incorporated with excipient and used in the form of ingestible tablets, buccal tablets, troches, capsules, elixirs, suspensions, syrups, wafers and the like.

The compounds may also be administered parenterally or intraperitoneally. Solutions of a compound can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (1990-18^{th} edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

The compounds may be administered to an animal alone or in combination with pharmaceutically acceptable carriers, as noted above, the proportion of which is determined by the solubility and chemical nature of the compound, chosen route of administration and standard pharmaceutical practice.

An effective amount of the compounds refers to the amount sufficient to promote myelopoiesis and can vary depending on many factors such as, in the case of administration in vivo the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. A CR compound may be administered initially in a suitable dosage that may be adjusted as required, depending on the clinical response.

The effects of a CR compound on myelopoiesis may be assessed using colony forming assays known in the art and as described in the examples. In one embodiment therefore, the methods of the invention include assessing effects of a CR compound on myelopoiesis.

For administration ex vivo, a CR compound can be administered to hematopoietic cells in a range from about 20-50 μM, for up to about 5 hours. It will be understood that in the case of administration to cells in vitro or ex-vivo, compounds may be added to the cells in culture, for example by adding the compounds to cells in culture, or by addition of culture medium containing the compounds to cells. Any cell culture medium which can support myelopoiesis may be used. Samples of cells may be obtained, using standard techniques, treated ex vivo, and introduced into a patient as is known in the art.

The compounds may be packaged as a kit and the invention in one aspect provides a kit comprising a CR compound and instructions for use of the compound, including to promote myelopoiesis and to treat neutropenia, aplastic anemia, or aplasia. In different embodiments, the kit may include a pharmaceutically acceptable carrier. The kit preferably includes CR4, CR11 or CR19.

All references cited herein are fully incorporated by reference.

### EXAMPLES

The CR compounds may be prepared as described in Examples 1 to 17.

Materials and Methods

¹H NMR spectra were obtained on a Varian Unity Plus spectrometer (USA) at 500 MHz with tetramethylsilane (TMS, Me₄Si) as an internal standard (δ=0). Electrospray mass spectra were recorded on an API III Plus triple quadrupole mass spectrometer (USA), with a direct introduction of the samples into the ionization source. Thin layer chromatography was performed with UV-254 aluminum-backed TLC sheets of 0.25 mm thickness (Kieselgel 60 F₂₅₄, Merck, Germany). HPLC separation of the compound of Example 13 was performed on a Waters 600 chromatograph (USA), column Nova-Pak C18 3.9×300 mm (Waters, USA). Vacuum distillations were done using Kugelrohr apparatus (Aldrich, USA) at stated temperatures of an oven. 3,5-Dimethoxy-4-hydroxycinnamaldehyde, 3,4-dimethoxycinnamic acid, 3,4-dihydroxycinnamic acid, 3,4-dimethoxybenzylamine, benzylamine, methyl cyanoacetate, were purchased from Aldrich (USA) and were used as received. The reagents were from Aldrich (USA). Solvents were purchased from Caledon (Canada).

### Example 1

N-(Cyanoacetyl)3,4-dimethoxybenzylaminocarbonyl (A₁)

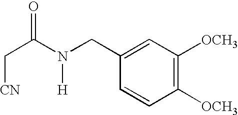

To 3,4-dimethoxybenzylamine (2.7 ml, 18 mmol) methyl cyanoacetate was added (1.6 ml, 18 mmol). The reaction was heated for 14 h at 100° C. Cooling gave a dark brown solid which was recrystallized from ethanol to give 2.90 g of the product (69% yield).

The product gave the following analytical data:

NMR (CD₃COCD₃, δ, ppm): 3.62 (s, 2H, CH₂CN), 3.78 (s, 6H, (OMe)₂), 4.34 (br.s., 2H, NHCH₂Ph), 6.84 (dd, 1H, J 1.95 and 8.1 Hz, H⁶), 6.88 (d, 1H, J 8.1 Hz, H⁵), 6.93 (d, 1H, J 1.95 Hz, H²), 7.80 (br.s., 1H, NH).

MS, m/e (rel. intensity, %): 235 (19) [M+H]⁺, 252 (100) [M+NH₄]⁺, 257 (33) [M+Na]⁺.

### Example 2

N-(Cyanoacetyl)3,4-dihydroxybenzylaminocarbonyl (A₂)

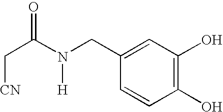

To N-(cyanoacetyl)3,4-dimethoxybenzylaminocarbonyl (Example 1, 0.2 g, 0.85 mmol) in 20 ml of CH₂Cl₂ boron tribromide was added under argon at −78° C. (0.24 ml, 2.56 mmol) in 2.5 ml of CH₂Cl₂. After 2 h the reaction was brought to room temperature and stirred overnight. The reaction was cooled to 0° C., 10 ml of 1N HCl was added, the solution was extracted with 3×50 ml of ethyl acetate, the organic phase was washed to neutral pH, dried with MgSO₄, and taken to dryness. The residue was purified by silica gel chromatography (CHCl₃—MeOH, 20:1) to give a yellow solid (0.07 g, 40% yield). The product gave the following analytical data:

NMR (CD₃COCD₃, δ, ppm): 2.83 (s, (OH)₂), 3.60 (s, 2H, CH₂CN), 4.25 (br.s., 2H, NHCH₂Ph), 6.63 (dd, 1H, J 1.95 and 8.1 Hz, H⁶), 6.75 (d, 1H, J 8.1 Hz, H⁵), 6.79 (d, 1H, J 1.95 Hz, H²), 7.71 (br.s., 1H, NH).

MS, m/e (rel. intensity, %): 207 (38) [M+H]⁺, 224(100) [M+NH₄]⁺, 229 (2.6) [M+Na]⁺.

### Example 3

(E,E)-2-(3,4-Dihydroxybenzylaminocarbonyl)-3-(3,5-dimethoxy-4-hydroxystyryl)acrylonitrile (CR11)

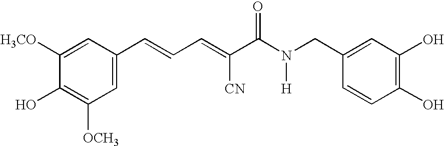

To 3,5-dimethoxy-4-hydroxycinnamaldehyde (0.042 g, 0.2 mmol) and N-(cyanoacetyl)3,4-dihydroxybenzylaminocarbonyl (Example 2, 0.042 g, 0.2 mmol) in 10 ml of ethanol 3 mg of β-alanine was added and the reaction was refluxed for 6 h. Water was added and the solid was recrystallized from 5 ml of ethanol twice to give 0.06 g (75%) of a red solid. The product gave the following analytical data:

NMR (CD₃COCD₃, δ, ppm): 2.81 (s, (OH)3), 3.89 (s, 6H, (OMe)₂), 4.39 (br.s., 2H, NHCH₂Ph), 6.68 (dd, 1H, J 1.95 and 8.1 Hz, H⁶′), 6.76 (d, 1H, J 8.1 Hz, H⁵′), 6.86 (d, 1H, J 1.95 Hz, H²′), 7.07 (br.s, 2H, H²⁺⁶), 7.16 (dd, 1H, J 11.7 and 15.1 Hz, PhCCHCCN olefinic), 7.37 (d, 1H, J 15.1 Hz, PhCH olefinic), 7.70 (br.s., 1H, NH), 7.98 (dd, 1H, J 0.75 and 11.7 Hz, CHCN olefinic).

MS, m/e (rel. intensity, %): 397 (100) [M+H]⁺, 414 (14) [M+NH₄]⁺.

### Example 4

N-(Cyanoacetyl)benzylaminocarbonyl (A₃)

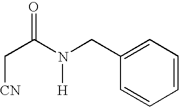

The compound was prepared as described in Example 1 by adding methyl cyanoacetate (1.3 ml, 14 mmol) to benzylamine (1.5 ml, 14 mmol). The compound was distilled in vacuo directly from the reaction mixture (Kugelrohr apparatus (Aldrich), 0.1 mm Hg, T. oven 180-190° C.) to give an off-white solid (2.34 g, 95%). The product gave the following analytical data:

NMR (CD₃COCD₃, δ, ppm): 3.39 (s, 2H, CNCH₂), 4.46 (d, 2H, J 5.4 Hz, NHCH₂Ph), 6.40 (br.s., 1H, NH), 7.24-7.36 (m, 5H, Ph).

MS, m/e (rel. intensity, %): 175 (64) [M+H]⁺, 192 [M+NH₄]⁺.

### Example 5

3,4-Dimethoxycinnamyl alcohol (A₆)

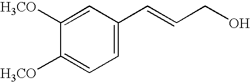

To a solution of 0.42 g (2.0 mmol) of 3,4-dimethoxycinnamic acid in 50 ml MeOH was added SOCl₂ (50 μl) and the mixture was stirred at 60° C. for 5 h. Methanol was taken to dryness and the obtained 3,4-dimethoxycinnamic acid methyl ester was reduced with 1M THF solution of diisobutylaluminum hydride (8.0 mmol) in absolute THF (50 ml) at 20° C. for 1 h. Water was added, the mixture was extracted with EtOAc, dried with MgSO₄ and distilled in vacuo (Kugelrohr apparatus (Aldrich), 0.1 mm Hg, T. oven 185-190° C.) giving an off-white solid, yield 0.36 g (92%), m.p. 70-71° C. The product gave the following analytical data:

NMR (CD₃COCD₃, δ, ppm): 3.77, 3.82 (2×s, 2×3H, OMe+OMe), 4.19 (d, 2H, J 5.0 Hz, CH₂OH), 6.25 (dt, 1H, J 5.0 and 15.5 Hz, PhCCH olefinic), 6.51 (d, 1H, J 15.5 Hz, PhCH olefinic), 6.89 (m, 2H, H⁵⁺⁶), 7.05 (br.s., 1H, H²).

MS, m/e (rel. intensity, %): 177 (100) [M−OH]+, 195 (4) [M+H]⁺, 212 (59) [M+NH₄ ]⁺, 217 (26) [M+Na]⁺.

### Example 6

3,4-Dimethoxycinnamaldehyde (A₇)

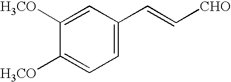

To a mixture of pyridinium dichromate (3.88 g, 10.3 mmol) and 4 g of finely grounded freshly activated molecular sieves 3 Å in 20 ml of CH₂Cl₂ 3,4-dimethoxycinnamyl alcohol in 10 ml of CH₂Cl₂ (Example 5, 1.00 g, 5.1 mmol) was added. The reaction was stirred for 2 h, 0.5 ml of methanol was added, the residue was passed through silica gel and washed with 300 ml of ethyl acetate. After evaporation the compound was purified by silica gel chromatography (hexane-EtOAc, 5:1) leading to a crystallizing oil (0.62 g, 63%).

The product gave the following analytical data:

NMR (CD₃COCD₃, δ, ppm): 3.90 (2×s, 2×3H, OCH₃+OCH₃), 6.70 (dd, 1H, J 7.6 and 16.0 Hz, PhC=CH olefinic), 7.05 (d, 1H, J 8.3 Hz, H⁵), 7.28 (dd, 1H, J 1.4 and 8.3 Hz, H⁶), 7.37 (d, 1H, J 1.4 Hz, H²), 7.60 (d, 1H, J 16.0 Hz, PhCH olefinic), 9.65 (d, 1H, J 7.6 Hz, CHO).

MS, m/e (rel. intensity, %): 193 (100) [M+H]⁺, 210 (26) [M+NH₄]⁺.

### Example 7

(E,E)-2-(Benzylaminocarbonyl)-3-(3,4-dimethoxystyryl) Acrylonitrile (CR2)

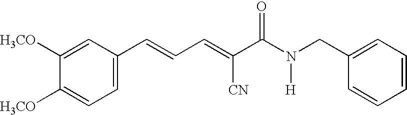

The compound was prepared as described in Example 3, by adding 3,4-dimethoxycinnamaldehyde (Example 6, 0.04 g, 0.2 mmol) to N-(cyanoacetyl)benzylaminocarbonyl (Example 4, 0.036 g, 0.2 mmol). After refluxing for 1 h and recrystallization from ethanol a yellow solid was obtained (0.045 g, 62%). The product gave the following analytical data:

NMR (CD₃COCD₃, δ, ppm): 3.90 (s, 2×3H, OMe+OMe), 4.57 (d, 2H, J<2 Hz, NHCH₂Ph), 7.08 (br.s., 1H, H²), 7.17 (dd, 1H, J 11.5 and 15.2 Hz, PhCCHCCN olefinic), 7.23-7.42 (m, 8H, aromatic+H⁵+H⁶+PhCH olefinic), 7.90 (br.t, 1H, NH), 8.05 (dd, 1H, J 0.55 and 11.5 Hz, CHCN olefinic).

### Example 8

(EE)-2-(Benzylaminocarbonyl)-3-(3,4-dihydroxystyryl)acrylonitrile (CR4)-Method A

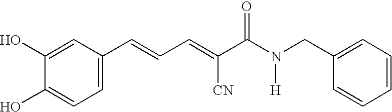

Boron tribromide (0.033 ml, 0.34 mmol) was added to (E,E)-2-(benzylaminocarbonyl)-3-(3,4-dimethoxystyryl)acrylonitrile (Example 7, 0.04 g, 0.11 mmol). The residue was purified by silica gel chromatography (CHCl₃—MeOH, 10:1) to give an orange solid (0.02 g, 55% yield). The product gave the following analytical data:

NMR (CD₃COCD₃, δ, ppm): 2.86 (br.s., 2H, (OH)₂), 4.55 (m, 2H, NHCH₂Ph), 6.90-7.42 (m, 10H, Ph+Ph′+olefinic), 7.87 (br.s., 1H, NH), 8.02 (dd, 1H, J<0.5 and 11.4 Hz, CHCN olefinic).

MS, m/e (rel. intensity, %): 295 (61) [M+H−CN]⁺, 321 (100) [M+H]^{+, 338} (30) [M+NH₄]⁺.

### Example 9

Methyl Ester of 3,4-bis(t-butyldimethylsilyloxy)cinnamic acid (A₈)

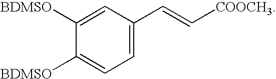

To a solution of 3.6 g (20 mmol) of 3,4-dihydroxycinnamic acid in 300 ml MeOH was added SOCl₂ (100 μl) and the mixture was stirred at 60° C. for 5 h. Methanol was taken to dryness and the obtained methyl ester was treated up with 10.2 g (68 mmol) of t-BuMe₂SiCl and 9.2 g (136 mmol) of imidazole in 100 ml DMF at 50° C. for 0.5 h. Mixture was diluted with water and extracted with hexane. Hexane was taken to dryness. The residue was distilled in vacuo (Kugelrohr apparatus (Aldrich), 0.1 mm Hg, T. oven 200-210° C.) and crystallized from hexane at −20° C. giving a white solid, yield 7.5 g(89%), m.p. 57-58° C. The product gave the following analytical data:

MS, m/e (rel. intensity, %): 423 (100) [M+H]⁺, 440 (98) [M+NH₄]⁺.

### Example 10

3,4-Bis(t-butyldimethylsilyloxy)cinnamyl alcohol (A₉)

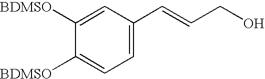

The compound was prepared as described in Example 5 by treating of 3,4-dihydroxycinnamic acid bis(BDMS) ether methyl ester (Example 9, 0.42 g, 1.0 mmol) with 1M THF solution of diisobutylaluminum hydride (4.0 mmol) in absolute ThF (25 ml) at 20° C. for 1 h. After distilling in vacuo (Kugelrohr apparatus (Aldrich), 0.1 mm Hg, T. oven 185-200° C.) a white viscous oil was obtained, yield 0.33 g (85%). The product gave the following analytical data:

NMR (CD₃COCD₃, δ, ppm): 0.23, 0.24 (2×s, 2×6H, Me₂Si+Me₂Si), 1.00, 1.02 (2×s, 2×9H, t-BuSi+t-BuSi), 4.19 (d, 2H, J 4.9 Hz, CH₂OH), 6.22 (dt, 1H, J 4.9 and 16.0 Hz, PhCCH olefinic), 6.49 (d, 1H, J 16.0 Hz, PhCH olefinic), 6.85 (d, 1H, J 8.2 Hz, H⁵), 6.92 (dd, 1H, J 2.1 and 8.2 Hz, H⁶), 6.97 (d, 1H, J 2.1 Hz, H²).

MS, m/e (rel. intensity, %): 377 (100) [M−OH]⁺, 395 (2) [M+H]⁺, 412 (15) [M+NH₄]³⁰ .

### Example 11

3,4-Bis(t-butyldimethylsilyloxy)cinnamaldehyde (A₁₀)

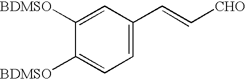

The compound was prepared as described in Example 6 by adding 3,4-bis(t-butyldimethylsilyloxy)cinnamyl alcohol (Example 10, 0.2 g, 0.5 mmol) in 5 ml of CH₂Cl₂ to a mixture of pyridinium dichromate (0.38 g, 1 mmol) and 1 g molecular sieves 3 Å in 20 ml of CH₂Cl₂. The residue was passed through silica gel and washed with 300 ml of EtOAc-hexane, 1:1. After evaporation the compound was purified by silica gel chromatography (hexane-EtOAc, 5:1) leading to an oil (0.15 g, 76%). The product gave the following analytical data:

NMR (CD₃COCD₃, δ, ppm): 0.26 and 0.28 (2×s, 2×6H, Me₂Si+Me₂Si), 1.01 and 1.02 (2×s, 2×9H, t-BuSi+t-BuSi), 6.60 (dd, 1H, J 7.7 and 15.9 Hz, PhCCH olefinic), 7.01 (dd, 1H, J<0.5 and 8.9 Hz, H⁶), 7.27 (m, 2H, H²⁺⁵), 7.60 (d, 1H, J 15.9 Hz, PhCH olefinic), 9.65 (d, 1H, J 7.7 Hz, CHO).

MS, m/e (rel. intensity, %): 367 (3) [M+H−CN]⁺, 393 (100) [M+H]⁺, 410 (10) [M+NH₄]⁺.

### Example 12

(E,E)-2-(Benzylaminocarbonyl)-3-(3,4-bis(t-butyldimethylsilyloxystyryl))acrylonitrile (CR18)

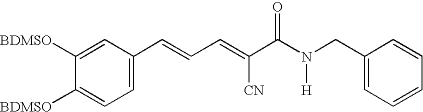

The compound was prepared as described in Example 3 by adding 3,4-bis(t-butyldimethylsilyloxy)cinnamaldehyde (Example 11, 0.100 g, 0.26 mmol) to N-(cyanoacetyl)benzylamide (Example 4, 0.044 g, 0.26 mmol. After refluxing for 2.5 h purification by silica gel chromatography (hexane-EtOAc, 15:1) provided a yellow solid (0.090 g, 64%). The product gave the following analytical data:

NMR (CD₃COCD₃, δ, ppm): 0.24 and 0.25 (2×s, 2×6H, Me₂Si+Me₂Si), 1.0 and 1.02 (2×s, 2×9H, t-BuSi+t-BuSi), 4.55 (br.s., 2H, NHCH₂Ph), 7.00 (d, 1H, J 8.5 Hz, H⁴), 7.12 (dd, 1H, J 11.7 and 15.6 Hz, PhCCHCCN olefinic), 7.24-7.43 (m, 8H, aromatic and olefinic protons), 7.93 (br.s., 1H, NH), 8.02 (dd, 1H, J<0.5 and 11.7 Hz, CHCN olefinic).

MS, m/e (rel. intensity, %): 523 (30) [M+H−CN]⁺, 540 (24) [M+NH₄−CN]⁺, 549 (89) [M+H]⁺, 566 (100) [M+NH₄]⁺.

### Example 13

(E,E)-2-(Benzylaminocarbonyl)-3-(3,4-dihydroxystyryl)acrylonitrile (CR4)-Method B

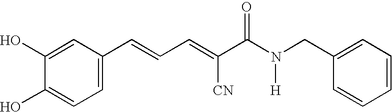

(E,E)-2-Benzylaminocarbonyl-3-[3,4bis(t-butyldimethylsilyloxystyryl)] acrylonitrile (Example 12, 0.028 g, 0.052 mmol) was treated with 60 μl of a 1M THF solution of tetra-n-butylammonium fluoride in 2 ml of dry THF for 0.5 h at 20° C. After evaporation the compound was dissolved in 5 ml of chloroform-methanol, 20:1, passed through silica gel and washed with chloroform-methanol, 20:1. The residue was purified by HPLC chromatography (MeCN—H₂O, 60:40, UV detection at 340 nm) leading to an orange solid (0.010 g, 62%). The analytical data were identical to the compound prepared as described in Example 8.

### Example 14

(E,E)-2-(3,4 Dihydroxybenzylaminocarbonyl)-3-styrylacrylonitrile (CR19)

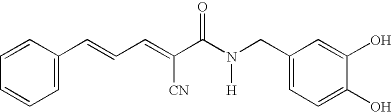

The compound was prepared as described in Example 3 by adding cinnamaldehyde (0.018 ml, 0.14 mmol) to N-(cyanoacetyl)3,4-dihydroxybenzylamide (Example 2, 0.03 g, 0.14 mmol). After refluxing for 2 h and recrystallization from ethanol, a yellow solid was obtained (0.027 g, 59%). The product gave the following analytical data:

NMR (CD₃COCD₃, δ, ppm): 2.82 (br.s., 2H, (OH)₂),4.39 (br.s., 2H, NHCH₂Ph), 6.70 (dd, 1H, J 1.9 and 8.2 Hz, H⁶′), 6.76 (d, 1H, J 8.2 Hz, H⁵′), 6.87 (d, 1H, J 1.9 Hz, H²′), 7.30 (dd, 1H, J 11.3 and 15.7 Hz, PhCCHCCN olefinic), 7.47 and 7.73 (2×m, 6H, aromatic protons and PHCH olefinic), 7.82 (br.s., 1H, NH), 8.04 (dd, 1H, J<0.5 and 11.3 Hz, CHCN olefinic).

MS, m/e (rel. intensity, %):321 (100) [M+H]⁺, 338 (65) [M+NH₄]⁺.

### Example 15

(E,E)-2-(Phenylethylamido)-3-(3,5-dimethoxy-4-hydroxystyryl)acrylonitrile (CR8)

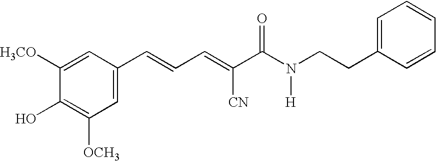

The compound was prepared as described in Example 3 by adding 3,5-dimethoxy-4-hydroxycinnamaldehyde (0.1 g, 0.48 mmol) to N-(cyanoacetyl)phenylethylamide (Example 29, 0.091 g, 0.48 mmol). The residue was purified by silica gel chromatography (CHCl₃-hexane, 1:1) to give a yellow solid (0.15 g, 83% yield). The product gave the following analytical data:

NMR (CD₃COCD₃, δ, ppm): 2.95 (t, 2H, J 7.6 Hz, CH₂Ph′), 3.62 (m, 2H, CH₂CPh′), 3.94 (s, 6H, (OMe)₂), 7.11 (s, 2H, H²⁺⁶), 7.19 (dd, 1H, J 11.7 and 15.3 Hz, PhCCHC olefinic), 7.23-7.36 (m, 5H, Ph′), 7.41 (d, 1H, J 15.3 Hz, PhCH olefinic), 7.45 (br.s., 1H, NH), 7.99 (d, 1H, J 11.7 Hz, CHCN olefinic).

MS, m/e (rel. intensity, %): 379 (100) [M+H]⁺, 396 (7) [M+NH₄]⁺.

### Example 16

Synthesis of (E,E)-2-cyano-3-(3,5-dimethoxy-4-hydroxystyryl)acrylonitrile (CR7)

The compound was obtained by the Knoevenagel condensation of 3,5-dimethoxy-4-hydroxycinnamaldehyde with malononitrile in ethanol in the presence of β-alanine (Scheme 1). The product was isolated from ethanol-water as a red powder. 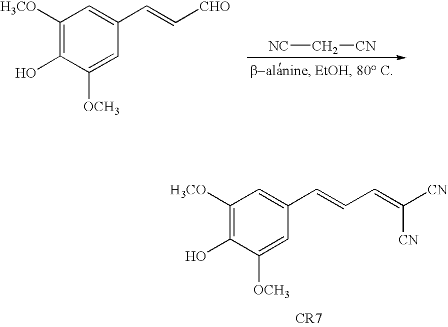
Materials and Methods

¹H NMR spectra were obtained on a Varian Unity Plus spectrometer at 500 MHz with Me₄Si as an internal standard (δ=0). Electrospray mass spectra were acquired on an API III⁺ triple quadrupole mass spectrometer (PE Sciex, Thornhill, Canada). Samples were directly introduced into the electrospray ionization source using an HPLC pump. Thin layer chromatography was performed with UV-254 aluminum-backed TLC sheets of 0.25 mm thickness (Kieselgel 60 F₂₅₄, Merck).

Malononitrile and 3,5-dimethoxy-4-hydroxycinnamaldehyde were purchased from Aldrich and were used as received. Solvents were purchased from Caledon (Canada).

(E,E)-2-cyano-3-(3,5-dimethoxy-4-hydroxystyryl)acylonitrile (CR7)

To the mixture of 42 mg (0.2 mmol) of 3,5-dimethoxy-4-hydroxycinnamaldehyde and 13 mg (0.2 mmol) of malononitrile in 2 mL EtOH, few crystals of β-alanine were added and the mixture was stirred at 80° C. for 4 h. 5 mL of water was added and the mixture was left at 0° C. for 2 h for crystallization. The product was filtered off, and washed with water. Yield 38 mg (75%). The product had the following analytical data:

¹H-NMR (δ, ppm): 3.89 (s, 6H, (OMe)₂), 7.16 (br.s, 2H, H2+6), 7.25 (dd, 1H, J 11.7 and 15.0 Hz, PhC═CH), 7.52 (d, 1H, J 15.0 Hz, PhCH═C), 7.98 (dd, 1H, J 0.77 and 11.7 Hz, CH═CCN). MS, m/e (rel.intensity, %): 257 (9) [M+H]⁺, 274 (100) [M+NH₄]⁺.

### Example 17

Synthesis of (E,E)-2-(benzylaminocarbonyl)-3-(3-methoxy-4-hydroxystyryl)acrylonitrile (CR56)

The compound was obtained by the Knoevenagel condensation of 3-methoxy-4-hydroxycinnamaldehyde with N-(cyanoacetyl)benzylamide (A3) in ethanol in the presence of equimolar amount of piperidine (Scheme). The product was isolated from ethanol-water as an orange powder and recrystallized from acetonitrile-water. 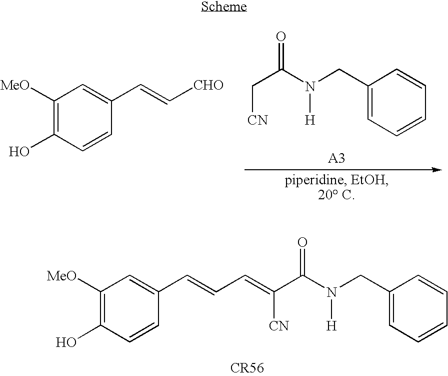
(E,E)-2-(Benzylaminocarbonyl-3-(3-methoxy-4-hydroxystyryl)acrylonitrile (CR56)

To a solution of 3-methoxy-4-hydroxycinnamaldehyde (36 mg, 0.2 mmol) and amide A3 (35 mg, 0.2 mmol) in 2 mL EtOH, 8 μl of piperidine (0.2 mmol) was added. The deep red solution was stirred at 20° C. for 1 h until the starting material disappeared. 2 mL 1N HCl were added followed by addition of 100 mL H₂O, and the mixture was kept at 0° C. for 2 h. The precipitated orange powder was washed with H₂O, recrystallized from MeCN—H₂O, and dried. Yield 48 mg (72%).

Example 18

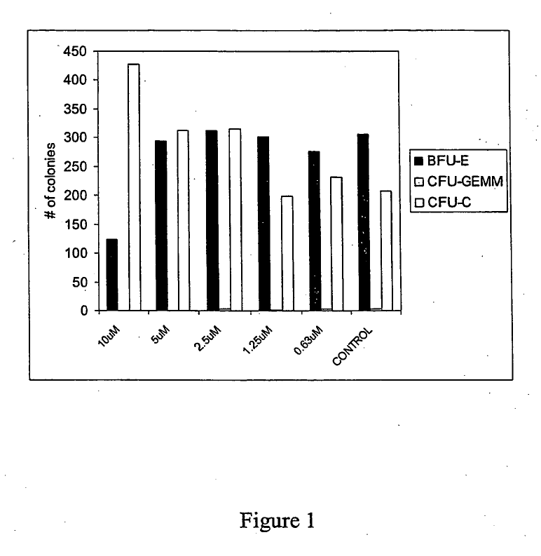

Treatment of normal bone marrow in culture with CR4. The CFU-GEMM assay was performed according to Fauser and Messner (Blood, 52(6) 1243-8,1978) and Messner and Fausser (Blut 41(5) 327-33, 1980) with some variations. In brief, heparinized bone marrow cells were layered over Percoll (1.077 gm/ml) and centrifuged at 400 g at 4° C. for 10 minutes to remove neutrophils and RBCs. The fractionated BM cells at 2×10⁵ cells/ml were cultured in IMDM containing 0.9% (vol/vol) methylcellulose supplemented with 30% FCS or normal human plasma, a cocktail of cytokines containing G-CSF (10 ng/ml), IL-3 (40 U/ml), MGF (50 ng/ml), Erythropoietin (2u/ml) or TPO (10 ng/ml) and 5×10⁻⁵M μ-2-mercaptoethanol. CR4 was added to the cells in concentrations indicated in FIG. 1 and the culture mixture was plated in 1 ml volumes into 35 mm petri dishes and incubated at 37° C., 5% CO₂ in a humidified atmosphere. All cultures were evaluated at 14 days for the number of BFU-E colonies (defined as aggregates of more than 500 hemaglobinized cells or, 3 or more erythroid subcolonies), CFU-C colonies (defined as granulocyte or monocyte-macrophage cells or both), and CFU-GEMM colonies (a mixed population comprising of all elements). All control samples in this and other examples were treated with the matching concentration of the solvent (DMSO) for the compounds in the same medium or in the case of Example 21, PBS, as indicated.

With reference to FIG. 1, CR4 displayed negligible toxicity upon normal bone marrow at doses up to 5 μM. CR4 stimulated CFU-C colony numbers at concentrations from about 0.6 to 10 μM. At 10 μM CR4 began to cause some inhibition of BFU-E colony formation, but at the same time significantly stimulated CFU-C colony numbers.

### Example 19

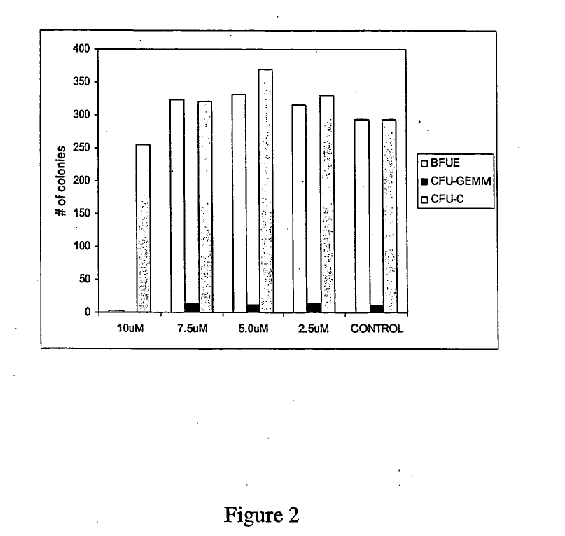

Treatment of CD34+ selected stem cells with CR4. Normal bone marrow cells were prepared and treated as in Example 15, except that CD34⁺ multipotent stem cells were selected for use from the complete bone marrow samples. Positive selection with anti-CD34 magnetic beads was utilized with the MACS magnetic cell sorting system (Miltenyi Biotec Inc, CA). As shown in FIG. 2, CR4 concentrations from 2.5-7.5 μM significantly increased the formation of CFU-C colonies from CD34⁺ stem cells.

### Example 20

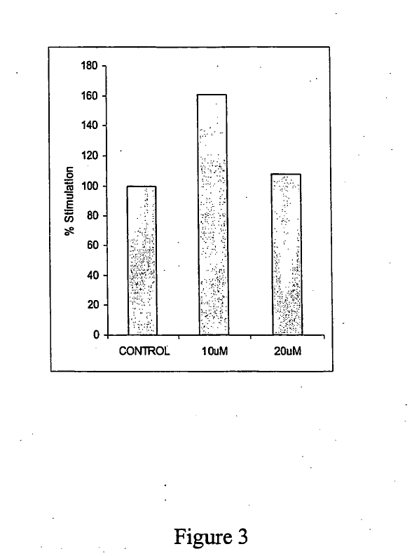

CFU-C stimulation with CR4 dose response. Bone marrow cells were prepared as in Example 15. CR4 was added to the cells at 10 or 20 μM. The culture mixture was plated in 1 ml volumes into 35 mm petri dishes and incubated at 37° C., 5% CO₂ in a humidified atmosphere. All cultures were evaluated at 14 days for the number of CFU-C colonies (defined as granulocyte or monocyte-macrophage cells or both). As shown in FIG. 3, 10 μM CR4 significantly increased CFU-C colony formation, but 20 μM did not and was equivalent to the control.

### Example 21

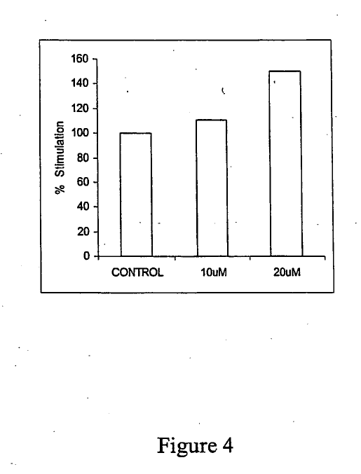

Stimulation of CFU-C colony formation by CR11. Bone marrow cells were prepared as in Example 15. CR11 was added to the cells at 10 or 20 μM. The culture mixture was plated in 1 ml volumes into 35 mm petri dishes and incubated at 37° C., 5% C₂ in a humidified atmosphere. All cultures were evaluated at 14 days for the number of CFU-C colonies. As shown in FIG. 4, 20 μM CR11 significantly increased the formation of CFU-C colonies, while 10 μM elevated colony numbers slightly.

### Example 22

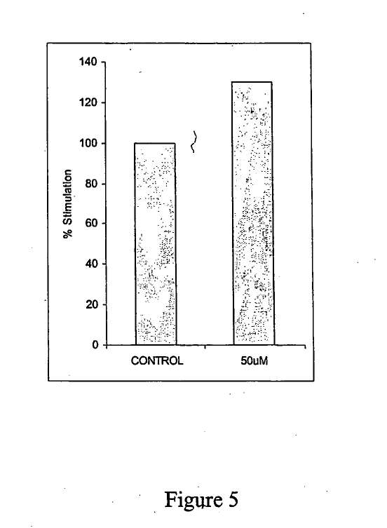

Stimulation of CFU-C colony formation with high dose CR4 for 2.5 hours. Bone marrow cells were layered over Percoll and centrifuged at 400 g at 4° C. for 10 minutes to remove neutrophils and RBCs. The cells were resuspended at 1×10⁶/ml in complete medium with or without 50 μM CR4. The cells were incubated with the compound for two and a half hours at 37° C., 5% CO₂. At the end of this period the cells were thoroughly washed in medium to remove CR4 and then cultured at 2×10⁵ cells/ml in IMDM containing 0.9% (vol/vol) methylcellulose supplemented with 30% FCS or normal human plasma, and a cocktail of cytokines containing G-CSF (10 ng/ml), IL-3 (40 U/ml,), MGF (50 ng/ml), Erythropoietin or TPO and 5×10⁻⁵M μ-2-mercaptoethanol. The culture mixture was plated in 1 ml volumes into 35 mm petri dishes and incubated at 37° C., 5% CO₂ in a humidified atmosphere. All cultures were evaluated at 14 days for the number of CFU-C colonies. As shown in FIG. 5, after only two and a half hours exposure to 50 μM CR4, CFU-C colony formation increased significantly.

### Example 23

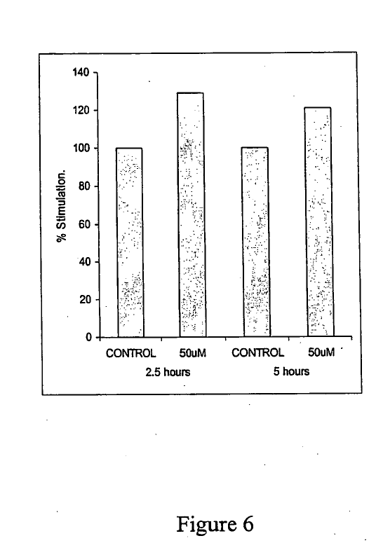

Stimulation of CFU-C colony formation with high dose CR4 for 2.5 and 5 hours. Bone marrow cells were prepared and treated as in Example 19, except exposure times of 2.5 and 5 hours were evaluated. The culture mixture was plated in 1 ml volumes into 35 mm petri dishes and incubated at 37° C., 5% CO₂ in a humidified atmosphere. All cultures were evaluated at 14 days for the number of CFU-GM colonies (defined as granulocyte or macrophage cells or both). As shown in FIG. 6, CFU-C colony formation increased significantly after both two and a half hours and five hours exposure to 5 μM CR4.

### Example 24

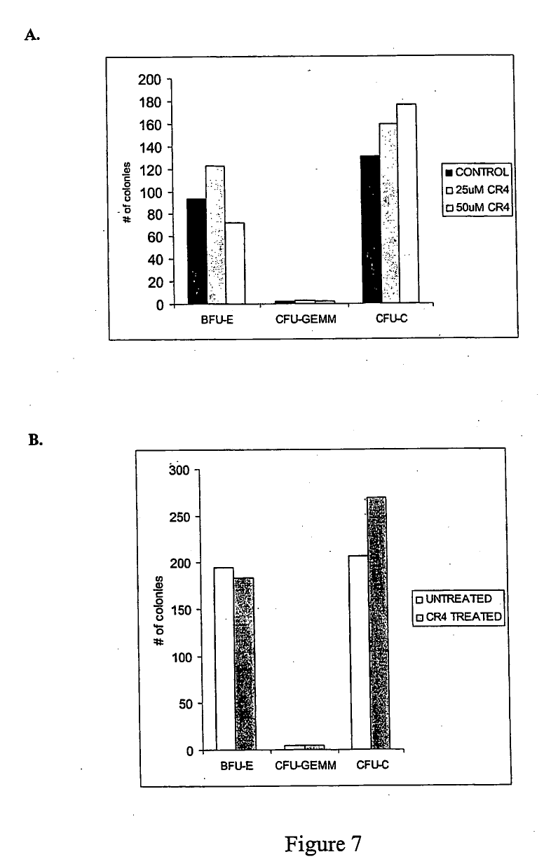

Stimulation of CFU-C colony formation with high dose CR4—two independent bone marrow samples (A&B). Bone marrow cells were prepared and treated as in Example 19, except that CR4 concentrations of 25 and 50 μM were evaluated. The culture mixtures was plated in 1 ml volumes into 35 mm petri dishes and incubated at 37° C., 5% CO₂ in a humidified atmosphere. All cultures were evaluated at 14 days for the number of CFU-C colonies. With reference to FIG. 7A, CFU-C colony formation increased significantly after five hours exposure to both 25 μM and 50 μM CR4. As shown in FIG. 7B, CFU-C colony formation in the other bone marrow sample increased significantly after five hours exposure to 50 μM CR4.

### Example 25

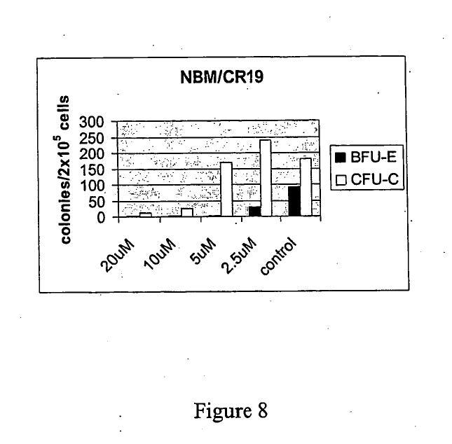

CFU-C stimulation with CR19 dose response. Bone marrow cells were prepared as in Example 15. CR19 was added to the cells at the concentrations indicated in FIG. 8. The culture mixture was plated in 1 ml volumes into 35 mm petri dishes and incubated at 37° C., 5% CO₂ in a humidified atmosphere. All cultures were evaluated at 14 days for the number of BFU-E and CFU-C colonies. As shown in FIG. 8, 2.5 μM CR19 significantly increased CFU-C colony formation.

### Example 26

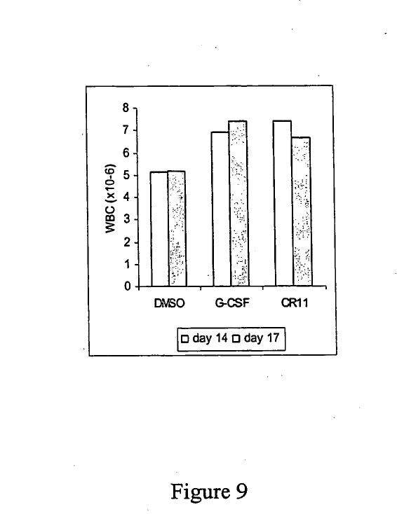

Promotion of peripheral white blood cell count by CR11. Mice were injected daily with CR11. 250 μl of 400 μM CR11 was injected in PBS. Controls were injected with matching concentrations of CR11 solvent (DMSO) in PBS, or recombinant human G-CSF. After 14 and 17 days blood peripheral blood samples were taken and white blood cell counts determined. Results from each group were averaged. As shown in FIG. 9, both G-CSF and CR11 significantly increased the white blood cell counts.

### Example 27

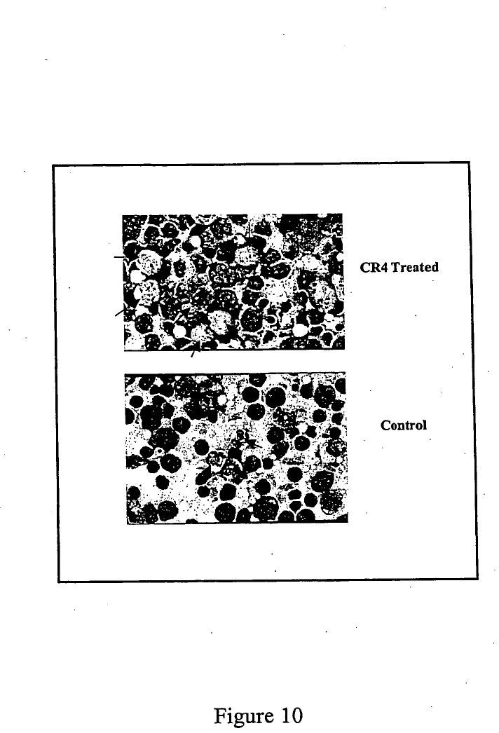

Increase in granulocytes in the spleen of CR4 treated mice. Mice were treated with CR4 for one month. CR4 was delivered from a subcutaneously implanted osmotic pump, with a resultant theoretical dosage of 200 μg/kg/hr. The pump was replaced weekly. Granulocytes are indicated by arrows in FIG. 10.

### Example 28

Long-term effects of CR4 (LTC-IC assay)

Materials and Methods

The long-term BM culture system provides the means to investigate the proliferation and differentiation properties of primitive hematopoietic cells. To assess the long-term effects of CR4 on early progenitor cells the long-term culture-initiating cell (LTC-IC) assay was set up utilizing stromal microenvironment. Briefly, to obtain stromal cells bone marrow mononuclear cells were suspended in long-term culture (LTC) media (MyeloCult Stem Cell Technologies Inc.) supplemented with 10⁻⁶ mol/L hydrocortisone sodium succinate (HSS; Sigma Aldrich Canada Ltd., Mississauga, Ont.,Canada). Approximately 1×10⁷ cells were plated into 25 cm² tissue culture flasks (Becton Dickinson Labware, Franklin Lakes, N.J., USA) and incubated for 3 days at 370° C. in a humidified atmosphere with 5% CO₂ followed by incubation at 33° C. Cultures were re-fed weekly by replacing half of the medium. Following stromal confluency (3-6 weeks) cells were trypsinized and irradiated (1,500 cGy) using a Cs source. Stromal cells were again replated in LTC media at a concentration of 0.5-1.0×10⁶ cells per 35 mm culture dishes (Nunc-Gibco BRL, Gaithersburg, Md.). Subcultured stroma was incubated for 2-3 days (at greater than 70% confluency) prior to co-culturing with fresh non-adherent overlay of hematopoietic cells.

Normal bone marrow mononuclear cells, purified by centrifugation on Percoll, at a concentration of 1×10⁶ cells/ml were pre-incubated with 50 uM CR4 or the drug diluent alone for a period of 5 hours at 37° C. in media. Cells were then washed twice to remove the compound and plated at 2×10⁵ cells/ml for the short-term CFU-GEMM clonogenic assay (as previously described) while the remainder of the non-adherent cells were co-cultured with normal stroma (as described above) for long-term cultures. Long-term cultures were set up with two unrelated normal bone marrow samples (NBM1 and NBM2). Half of the non-adherent mononuclear cells were removed weekly from the stromal co-cultures and plated for colony-forming cells (CFU-GM) in the CFU-GEMM assay.

Results

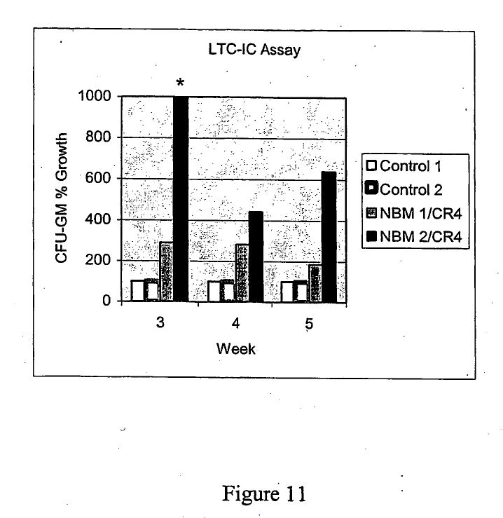

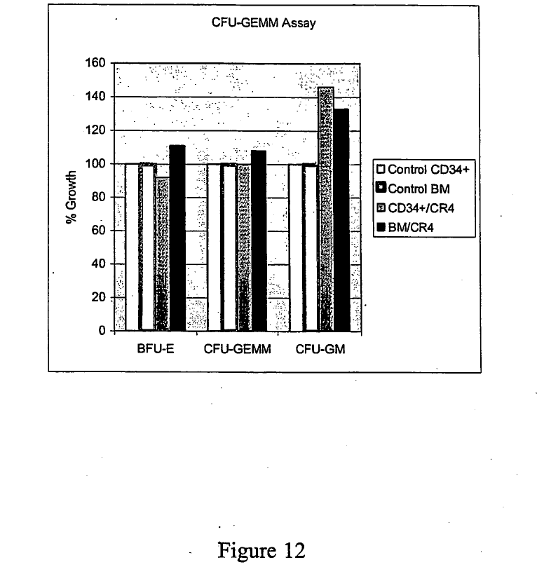

The long-term cultures on both NBM1 and NBM2 demonstrated a significant increase in myeloproliferation (FIG. 11) when pre-incubated with CR4. A greater degree of myeloproliferation was observed in the long-term cultures than in the short term CFU-GEMM assay (FIG. 12). All colonies were of normal morphology and demonstrated cytokine growth dependency. FIG. 12 also demonstrates that myeloproliferation can be induced with purified CD34+ and hematopoietic stem cells.

### Example 29

(Cyanoacetyl)phenylethylamide (A4)

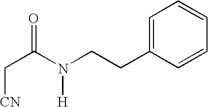

The compound was prepared as described in Example 1 by adding methyl cyanoacetate (1.1 ml, 12.4 mmol) to phenylethylamine (1.55 ml, 12.4 mmol). The compound was distilled in vacuo directly from the reaction mixture (Kugelrohr apparatus (Aldrich), 0.1 mm Hg, T. oven 190-195° C.) to give an off-white solid (2.14 g, 91%). The product gave the following analytical data:

NMR (CD₃COCD₃, δ, ppm): 2.80 (t, 2H, J 7.6 Hz, PhCH₂), 3.46 (br.t, 2H, J 7.6 Hz, PhCCH₂), 3.54 (s, 2H, CNCH₂), 7.20-7.31 (m, 5H, Ph), 7.51 (br.s., 1H, NH).

MS, m/e (rel. intensity, %): 189 (100) [M+H]⁺, 206 (99) [M+NH₄]⁺.

References.

1. Lakshman R., and A. Finn. 2001. Neutrophil disorders and their management. J Clin Pathol 54:7.
2. Crokaert, F. 2000. Febrile neutropenia in children. Int J Antimicrob Agents 16:173.
3. Adams, V. R. 2000. Adverse events associated with chemotherapy for common cancers. Pharmacotherapy 20:96S.
4. Tsiodras, S., G. Samonis, M. J. Keating, and D. P. Kontoyiannis. 2000. Infection and immunity in chronic lymphocytic leukemia. Mayo Clin Proc 75:1039.
5. Greene, J. N., D. C. Linch, and C. B. Miller. 2000. Current treatments for infection in neutropenic patients with hematologic malignancy. Oncology (Huntingt) 14:31.
6. Prentice, A. G., and P. Donnelly. 2001. Oral antifungals as prophylaxis in haematological malignancy. Blood Rev 15:1.
7. Germeshausen, M., M. Ballmaier, H. Schulze, K. Welte, T. Flohr, K. Beiske, I. Storm-Mathisen, and T. G. Abrahamsen. 2001. Granulocyte colony-stimulating factor receptor mutations in a patient with acute lymphoblastic leukemia secondary to severe congenital neutropenia. Blood 97:829.
8. Germeshausen, M., M. Ballmaier, and K. Welte. 2001. Implications of mutations in hematopoietic growth factor receptor genes in congenital cytopenias. Ann N Y Acad Sci 938:305.
9. Wagner, L. M., and W. L. Furman. 2001. Haemopoietic growth factors in pediatric oncology: a review of the literature. Pediatric Drugs 3:195.
10. Smith, M. A., and J. G. Smith. 2001. The use of granulocyte colony-stimulating factor for treatment of autoimmune neutropenia. Curr Opin Hematol 8:165.
11. Pecora, A. L. 2001. Progress in clinical application of use of progenitor cells expanded with hematopoietic growth factors. Curr Opin Hematol 8:142.
12. Lieschke, G. J., D. Grail, G. Hodgson, D. Metcalf, E. Stanley, C. Cheers, K. J. Fowler, S. Basu, Y. F. Zhan, and A. R. Dunn. 1994. Mice lacking granulocyte colony-stimulating factor have chronic neutropenia, granulocyte and macrophage progenitor cell deficiency, and impaired neutrophil mobilization. Blood 84:173 7.
13. Hermans, M. H., A. C. Ward, C. Antonissen, A. Karis, B. Lowenberg, and I. P. Touw. 1998. Perturbed granulopoiesis in mice with a targeted mutation in the granulocyte colony-stimulating factor receptor gene associated with severe chronic neutropenia. Blood 92:32.
14. Hubel, K, D. C. Dale, A. Engert, and W. C. Liles. 2000. Use of G-CSF for granulocyte transfusion therapy. Cytokines Cell Mol Ther 6:89.
15. Hubel, K., D. C. Dale, A. Engert, and W. C. Liles. 2001. Current status of granulocyte (neutrophil) transfusion therapy for infectious diseases. J Infect Dis 183:321.
16. Yigal Dror and Melvin H Freedman: Shwacbman-Diamond Syndrome: An Inherited Preleukemia Bone Marrow Failure Disorder with Aberrant Hematopoietic Progenitors and Faulty Marrow Microenvironment. Blood, Vol. 94 #9 (Nov 1), 1999, pp3048-3054.
17. Olga I Gan, Barbara Murdoch, Andre Larochelle and John E. Dick: Differential Maintenance of Primitive Human SCID-Repopulating Cells, Clonogenic Progenitors and Long-Term Culture-Initiating Cells After Incubation on Human Bone Marrow Stromal Cells. Blood, Vol. 90 #2 (July 15), 1997 pp 641-650.

1-23. (canceled)

24. A method of promoting myelopoiesis comprising administering an effective amount of a CR compound to hematopoietic cell or an animal in need thereof.

25. The method according to claim 24 wherein the CR compound is CR4, CR11 or CR19.

26. The method according to claim 25 wherein hematopoietic cell is bone marrow cell or peripheral blood stem cell.

27. The method according to claim 25 wherein the animal is human

28. The method according to claim 27 wherein the human is suffering from or at risk of neutropenia, aplastic anemia or aplasia.

29. The method of claim 28 wherein neutropenia is chemotherapy or drug induced neutropenia or neutropenia secondary to malignancy.

30. The method according to claim 29 wherein the malignancy is G-CSF responsive malignancy.

31. The method according to claim 27 wherein the human is a donor of bone marrow cell or peripheral blood stem cell.

32. The method according to claim 27 wherein the human has received or is in need of bone marrow cell or peripheral blood stem cell transplant.

33. A method of treating a patient suffering from or at risk of neutropenia, aplastic anemia or aplasia comprising administering an effective amount of a CR compound to the patient.

34. The method according to claim 33 wherein the CR compound is CR4, CR11 or CR19.

35. The method according to claim 34 wherein neutropenia is chemotherapy or drug induced neutropenia.

36. The method according to claim 35 further comprising the step of transplanting bone marrow cell or peripheral blood stem cell into the patient wherein CR4, CR11 or CR19 is administered to the patient before or after the step of transplanting.

37. The method according to claim 36 further comprising administering CR4, CR11 or CR19 ex vivo to bone marrow cell or peripheral blood stem cell prior to transplanting.

38. The method according to claim 37 wherein bone marrow cell or peripheral blood stem cell is autologous.

39. The method according to claim 34 wherein neutropenia is secondary to malignancy.

40. The method according to claim 39 wherein the malignancy is G-CSF responsive malignancy.

41. A method of promoting myelopoiesis of hematopoietic cell ex vivo comprising administering an effective amount of a CR compound to the hematopoietic cell.

42. The method according to claim 41 wherein the CR compound is CR4, CR11 or CR19.

43. The method according to claim 42 wherein an amount of about 20-50 μM is administered to up to 5 hours.

44. The method according to claim 43 wherein the hematopoietic cell is bone marrow cell or peripheral blood stem cell of a donor.

45. The method according to claim 44 wherein the hematopoietic cell is bone marrow cell or peripheral blood stem cell of a patient in need of autologous bone marrow or peripheral blood stem cell transplant.

46. A method of treating a patient suffering from or at risk of neutropenia, aplastic anemia or aplasia comprising introducing hematopoietic cell to the patient wherein a CR compound has been administered to the cell ex-vivo in an amount effective to promote myelopoiesis.

47. The method according to claim 46 wherein the CR compound is CR4, CR11 or CR 19.

48. The method according to claim 47 wherein the hematopoietic cell is bone marrow cell or peripheral blood stem cell.

49. The method according to claim 48 wherein the hematopoietic cell is autologous.

50. A kit comprising a CR compound and instructions for use to promote myelopoiesis.

51. A kit comprising a CR compound and instructions for use to treat neutropenia, aplastic anemia or aplasia.

52. The kit according to claim 50 wherein the CR compound is CR4, CR11 or CR19.

53. The kit according to claim 51 wherein the CR compound is CR4, CR11 or CR19.

54. The kit according to claim 52 comprising instructions to promote myelopoiesis of hematopoietic cell ex-vivo.

55. The kit according to claim 54 further comprising instructions to administer 50 μM of CR4, CR11 or CR19 to the hematopoietic cell for up to about 5 hours.
